(19) United States
(12) Patent Application Publication
(10) Pub. No.: US 2008/0154374 A1
(43) Pub. Date: Jun. 26, 2008

(54) JOINT IMPLANT AND A SURGICAL METHOD ASSOCIATED THEREWITH

(76) Inventor: ROBERT DAVID LABROM, TARINGA (AU)

Correspondence Address: MICHAEL WINFIELD GOLTRY, 4000 N. CENTRAL AVENUE, SUITE 1220, PHOENIX, AZ, 85012, US

(21) Appl. No.: 11/958,581

(22) Filed: Dec. 18, 2007

Related U.S. Application Data
(60) Provisional application No. 60/875,974, filed on Dec. 20, 2006.
(60) Provisional application No. 60/909,056, filed on Mar. 30, 2007.

(51) Int. Cl. A61F 2/44; A61B 17/58

(52) U.S. Cl. 623/17.12; 623/17.11; 606/79

(57) ABSTRACT

A method of performing surgery to enable joint fusion by preparing bony surfaces of a joint to create an enlarged space between sides of the joint in which subchondral bone of the joint is exposed, inserting a hollow structural implant, having at least two large fenestrations which are located on substantially opposite sides of the implant into the enlarged space so that the implant contacts the subchondral bone and orientating the implant so that the large fenestrations are located adjacent the subchondral bone on respective sides of the joint.

### CROSS REFERENCE TO RELATED APPLICATIONS

This application claims the benefit of U.S. Provisional Application Ser. No. 60/875,974, filed 20 Dec. 2006, and U.S. Provisional Application Ser. No. 60/909,056, filed 30 Mar. 2007.

### FIELD OF THE INVENTION

This invention relates to a joint implant and a surgical technique associated therewith. In particular the invention relates to spinal facet joint fusion and therefore will be described in this context. However, it should be appreciated that the implant may be used for fusing other joints throughout the body such as the radio-carpal joint, acromio-clavicular joint, carpal joints, metacarpal joints, tarsal joints, or any other synovial or fibrous joint in the skeleton.

### BACKGROUND OF THE INVENTION

Spinal fusion is a very common procedure performed via posterior surgical approaches for degenerative and deformity spinal pathologies. Spinal fusion can also address fusion of spinal levels adjacent to motion retaining devices/techniques. Spinal fusion limits motion between adjacent vertebrae to help eliminate pain arising from vertebrae applying pressure to a nerve root or neural element.

Typically posterior spinal fusion is achieved by inter-transverse process spinal fusion. This surgical technique often involves the placement of pedicle screws within vertebral bone and then attaching associated rods to associated pedicle screws. The pedicle screws in combination with the rods provide stability to the vertebrae so that bone graft can be placed between adjacent transverse processes and bone growth can occur to create permanent fusion of the spine.

Inter-transverse process spinal fusion is morbid with open approach surgical techniques. Accordingly, morbidity is reduced using more minimally invasive techniques to approach the posterior spinal elements. Further, bone graft delivery, containment, ectopic bone formation—especially with liquid bone morphogenic protein like substances, and resorption of loose bone graft remain problems with inter-transverse process spinal fusion.

Historically, posterior spinal fusions have also used a technique known as a Moe fusion (described by Dr John Moe). The surgical technique involves a partial destruction of the bony facet joint, decortication of surrounding bone surfaces, and insertion of non-structural bone chips/pieces into a space made after removal of the cartilage surfaces of the facet joint. There has even been the suggestion of surgical partial ablation of the joint with the use of an osteotome, gouge or bone nibbler.

This technique is not as frequently used today and the triple joint complex (i.e., the intervertebral disc space and the two facet joints) being fused may be biomechanically destabilised because of a space created between the facet joint surfaces, or worse, by the subtotal resection of the entire bony facet joint complexes. This technique leads to increased load sharing on any associated pedicle screw/rod construct and therefore may lead to increased loosening of such devices, and reduced fusion rates. However, there have been some advances in spinal facet fusions techniques.

US Patent Application No. 20060111782 and 20060111779 in the name of Petersen disclose minimally invasive spinal facet joint fusion. In particular, the patent applications disclose a facet joint fusion system that utilises a punch or drill that creates a hole through both sides of the spinal facet joint in a conical pattern. The hole is then filled with either the patients own harvested and compacted bone plug using iliac crest autograft, pre-made, pre-shaped cortical cadaveric allograft or pre-made, pre-shaped synthetic grafts.

The above technique works well in assisting in spinal facet joint fusion. However, the hole created in the spinal facet joint and filled by the bone plug may not be stable enough after surgery. The bone plug is relatively soft and therefore is able to be crushed with relative movement of the spinal facets. The minimisation of the hole created by compression of the bone plug may cause nerve compression which is undesirable. Pedicle screws and rods are therefore often required with this type of surgery and loosening of the screws in the pedicles in this setting would be undesirable and probable.

US Patent Application No. 20060085068 in the name of Barry discloses spinal facet joint implants and an associated method of non-invasive surgery to locate these implants within a spinal facet joint. The method includes the use of a guide wire to locate the implants in position within a spinal facet joint. Subsequently, each of the spinal facet joints has a hole that extends through the spinal facet joints. Hence, any application of a bone growth media to the implants to promote fusion has the potential to pass through the hole in the implant onto the underlying nerve root. This can cause damage to the nerve root which is undesirable.

US Patent application No. 20040111093 and 20060111782 in the name of Chappuis disclose a facet fusion system. In particular, the discloser relates to tapered implants placed within a surgically prepared spinal facet joint. The spinal facet joint system works reasonably well. However, the facet joint fusion time is relatively high as there are a limited number of fenestrations that extend through the implants that promote fusion. Further, many of the implants are solid which do not permit osteoinductive agents to be placed within the implants.

It is an object of the invention to overcome or alleviate one or more of the above disadvantages or provide the consumer with a useful or commercial choice.

### SUMMARY OF THE INVENTION

In one form, although not necessarily the only or broadest form, the invention resides in a method of performing surgery to enable joint fusion the steps including:

preparing bony surfaces of a joint to create an enlarged space between sides of the joint in which subchondral bone of the joint is exposed;

inserting a hollow structural implant, having at least two large fenestrations which are located on substantially opposite sides of the implant, into the enlarged space so that the implant contacts the subchondral bone; and

orientating the implant so that the large fenestrations are located adjacent the subchondral bone on respective sides of the joint.

Preferably, once the implant is located within the joint, a hollow cavity of the implant is filled with an oesteoconductive agent so that the oesteoconductive agent contacts the subchondral bone surfaces through the large fenestrations. An osteoinductive agent may also be added to the implant and be contained within a sponge. The sponge may be compressed within the implant. The graft composite within the hollow implant may contain any osteoinductive material such as bone morphogenic protein, or similar.

The oesteoconductive agent may include bone graft material eg. autograft, allograft, bone mineral substitute (TCP—tricalcium phosphate, BCP—bicalcium phosphate, HA—hydroxyapatite).

The surgical steps may be performed in an open or minimally invasive environment. The surgical steps may include utilising computerized and/or combined fluoroscopic navigation to assist in accurate placement of the trial or final implants.

The bony surfaces of the spinal facet joint may be manually and/or mechanically prepared. The preparation of the bony surfaces may include burring, drilling, taping, rasping, broaching and/or reaming.

Preferably, milling of the bony surfaces of the joint is performed to obtain a bone hole. The orientation of the bone hole may be made through a highly variable range of trajectories relative to the plane of an articular surface of the joint. The trajectory may be varied from parallel to the articular surface of the joint through to perpendicular to the articular surface of the joint.

The patient may be moved to a surgical position to distract the joint.

The implant may be inserted via a driving force. Alternatively, the implant may be inserted using a rotational force.

The implant may distract and fuse the joint.

In yet another form, the invention resides in an implant able to be inserted into a surgically prepared joint space, the implant including:

a body having at least one large fenestrations extending through the body; and

at least one barb extending outwardly from a periphery of the body.

Preferably, the implant is made from and/or coated with material that promotes bone growth such as hydroxyapatite, or a roughened external surface that promotes bone on-growth.

Normally the body of the implant is frusto conical in shape. The body may have a hollow central cavity to receive osteoconductive or osteoinductive agents. The body may have an end wall to hold the osteoconductive or osteoinductive agent within the implant.

Preferably, the body includes a skirt that extends around the body adjacent the distal end of the body.

Preferably, the body has at least two large fenestrations which are located on substantially opposite sides of the body.

The fenestrations may be sized to have an external surface area of at least 35% of the total external surface area of the implant. Preferably, the fenestrations may be sized to have an external surface area of at least 50% of the total external surface area of the implant. More preferably, the fenestrations may be sized to have an external surface area of at least 65% of the total external surface area of the implant, though a range of 35% to 70% will be likely.

Scraping holes may be located through the body adjacent the barb. The barb may be shaped to scape bone material into the central cavity when the implant is rotated.

Preferably, the implant is tapered. The implant may be of various shapes and could be trapezoidal, ovoid, cylindrical, or any other shape.

One or more channels may extend along an internal wall of the body.

The implant may be constructed from materials including PEEK™ (oxy-1,4-phenyleneoxy-1,4-phenylene-carbonyl-1,4-phenylene), carbon fiber, metals such as titanium, stainless steel, chrome cobalt, and Nitinol, elastomer, silicone, bone cement, or plastics, TCP—tricalcium phosphate, BCP—bicalcium phosphate, HA—hydroxyapatite or combination of the above.

The implant may be made of a material and/or have design features that permit a degree of motion to occur through or around the implant such that it permits an environment suitable for dynamic fusion. Such an implant may be used in combination with dynamic posterior fusion constructs.

The implant may have any combination of holes or pores or gaps that permits bone to grow through the device and the easy passage of osteoinductive agents.

### BRIEF DESCRIPTION OF THE DRAWINGS

Embodiments of the invention will be described with the reference to the accompany drawings in which:

FIG. 1A is a perspective view of a first embodiment facet joint implant;

FIG. 1B is a side view of the facet joint implant of FIG. 1A;

FIG. 1C is an end view of the facet joint implant of FIG. 1A;

FIG. 2A is a perspective view of an implant tool used with the spinal facet joint implant of FIG. 1A;

FIG. 2B is a perspective view of the implant tool of FIG. 11A engaging the spinal facet joint implant of FIG. 1A;

FIG. 3A is a perspective view of a variation of the implant tool of FIG. 2A;

FIG. 3B is a perspective view of the implant tool of FIG. 3A engaging the spinal facet joint implant of FIG. 1A;

FIG. 4A is a plan view of a spinal facet joint;

FIG. 4B is a plan view of a surgically prepared spinal facet joint;

FIG. 4C is a plan view of a spinal facet joint with implant;

FIG. 4D is a plan view of a spinal facet joint with rotated implant;

FIG. 5A is a side view of a second embodiment spinal facet joint implant;

FIG. 5B is a side sectional view of the spinal facet joint implant of FIG. 5A;

FIG. 5C is a top sectional view of the spinal facet joint implant of FIG. 5A;

FIG. 5D is a perspective view of the spinal facet joint implant of FIG. 5A;

FIG. 6A is a side view of a graft material impaction tool engaging the spinal facet joint of FIG. 5A; and

FIG. 6B is a side sectional view of a graft material impaction tool engaging the spinal facet joint of FIG. 5A.

### DETAILED DESCRIPTION OF THE PREFERRED EMBODIMENT

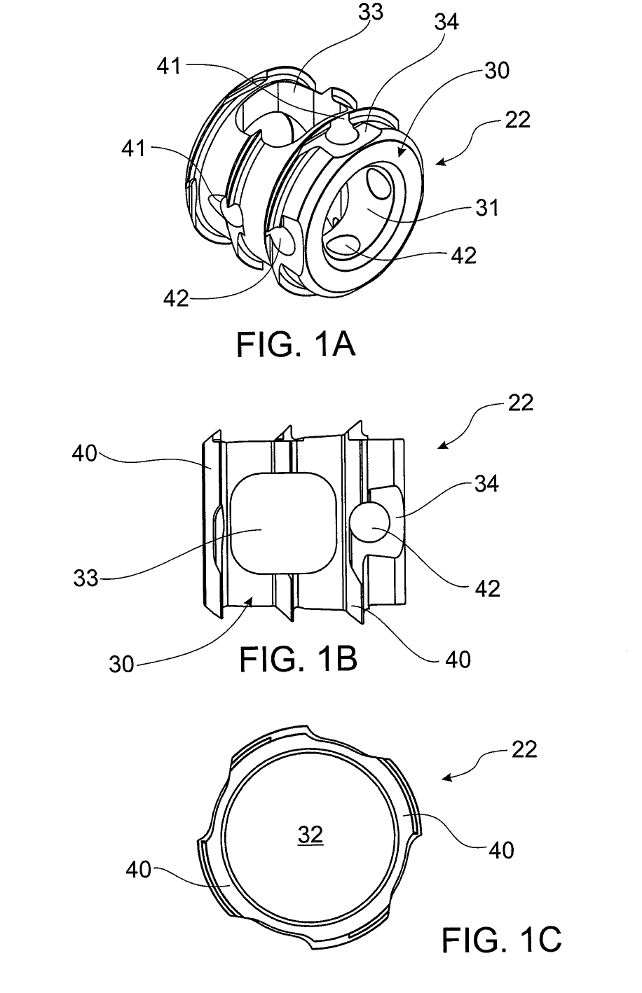

FIGS. 1A to 1C show an implant 22 able to be inserted into a surgically prepared spinal facet joint. It should be appreciated that even though this implant 22 has been specifically developed for use in surgically prepared spinal facet joints, it may have applications in other areas of the body such as the radio-carpal joint, acromio-clavicular joint, carpal joints, metacarpal joints, tarsal joints, or any other synovial or fibrous joint in the skeleton.

The implant 22 is made from titanium and may be coated with hydroxyapatite, or treated with a roughening technique such as acid/alkali treatments to promote a surface that enables bone on-growth. The implant 22 includes body 30 which is frusto conical in shape. That is, the body 30 is tapered from a top of the body 30 to a base of the body 30. A hollow central cavity 31 extends through a centre of the body 30 and an end wall 32 is located adjacent the end of the body 30. Large fenestrations 33 extend through the body 30. The large fenestrations 33 have an external surface area ratio of approximately 65% of the total external surface area of implant 22. A series of circumferential, spaced-apart, barbs 40 are located along the length of the body 30. Each barb 40 has a scraping face 41 which is able to engage with the spinal facet joint. A series of scraping holes 42 are located adjacent some of the scraping faces 41 of some of the barbs 40. The barbs 40 are shaped so that rotation of the barb 40 will cause the scraping face 41 to engage or scrape the spinal facet joints upon rotation of the body 30. A number of flat sections 34 are located adjacent the top of the body 30. The flats sections 34 are used to rotate the body 30.

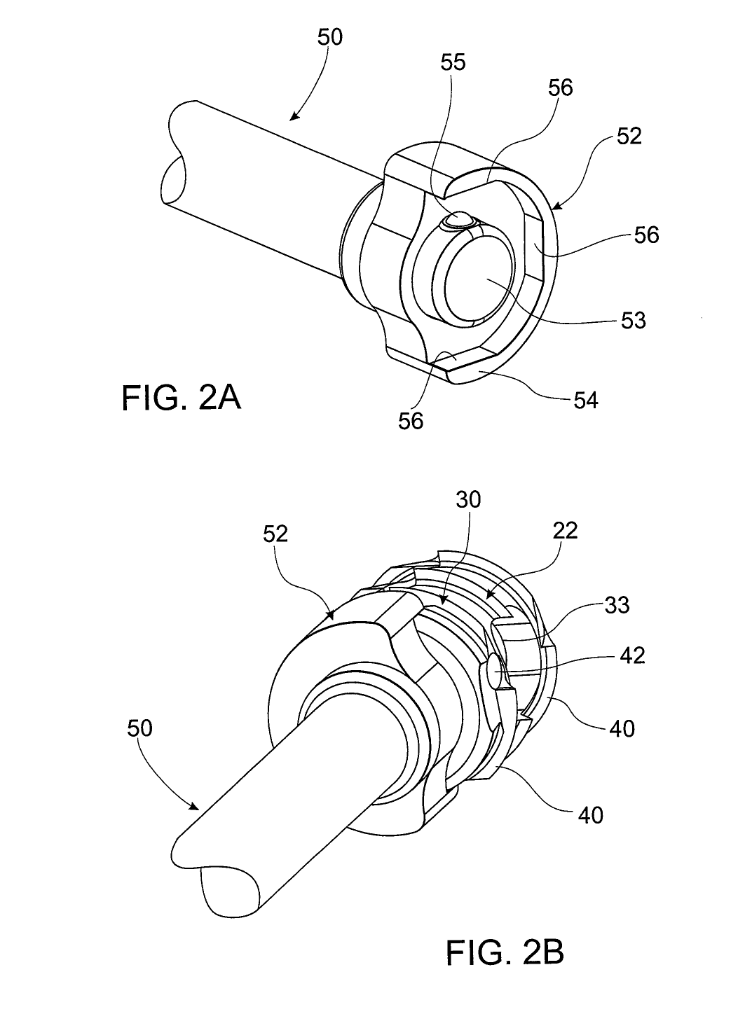

FIGS. 2A and 2B show an implant tool that is used to implant the implant shown in FIGS. 1A to 1C. The implant tool 50 includes a handle (not shown) and a head 52. The handle is gripped by a user and is able to be both rotated and driven. The head 52 is used to engage the top of the body 30 of the implant 22. The head 52 has a central boss 53 and an outer ring 54. A depressible ball 55 is located on an edge of the boss 53. The outer ring 54 has a series of flat sections 56 located along an inner circumference of the outer ring 54.

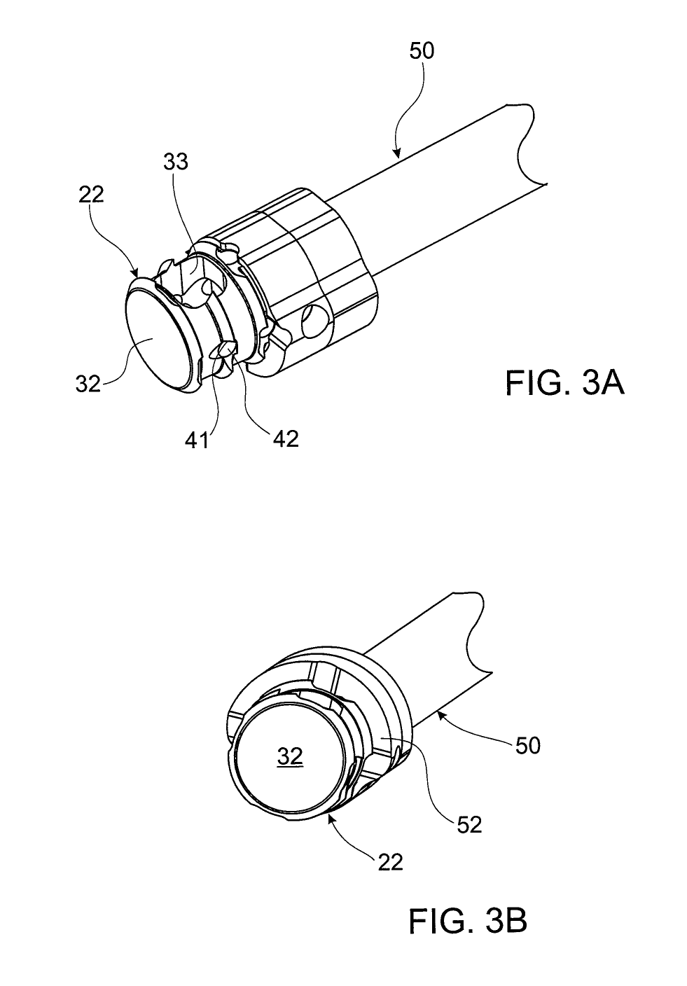

FIGS. 3A and 3B show a variation of the implant tool 50. This implant tool 50 has a head 52 that has a diameter larger than the diameter of the body 30. This oversized head 52 prevents the implant 22 being driven past the top of the spinal facet joint and contacting the nerve root located directly below the spinal facet joint.

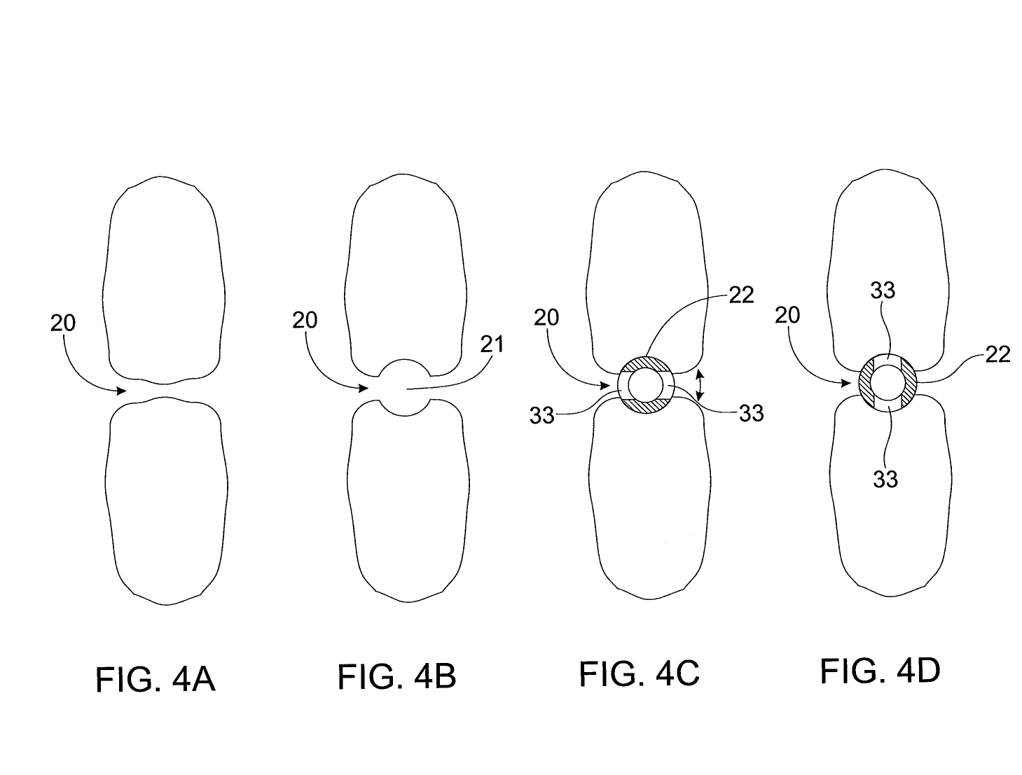

FIG. 4A a spinal facet joint 20 that is required to be biologically fused in a surgical procedure. The facet joint 20 allows articulation between the vertebrae.

The surgical procedure commences by placing the patient prone or in a lateral position on an operating table. The skin and the deeper muscle layers of the patent are incised in a typical manner to partially expose the two spinal vertebrae 10 so that access is provided to a facet joint 20. It should be appreciated that minimally invasive surgical techniques may be utilised. The patient may be placed in a forward flexed lateral lying position to distract the spinal facet joints 20.

Further, the spinal facet joint 20 is further distracted using a distraction tool such as a double-action interspinous process manual distracter tool. Alternatively or additionally, an interspinous process spacer implant may be placed between the spinous processes of the inter-vertebral level to hold open the spinal facet joint.

It should be appreciated that moving the patient to a forward flexed lateral lying position and/or using a distraction tool and/or interspinous process spacer may not be necessary if the spinal facet joints are sufficiently distracted to provide access to the bony surfaces of the spinal facet joints.

Once the spinal facet joints 20 are distracted somewhat, preparation of the bony surfaces of the spinal facet joints is commenced. Preparation involves burring, drilling, taping, rasping, broaching and/or reaming the bony surfaces of each spinal facet joint 20 to create an enlarged spinal facet space 21 as shown in FIG. 4B. It should be appreciated that preparation of each spinal facet joint 20 may be manually conducted or may use standard mechanical surgical tools such as a pneumatic drill or bone mill. Burring, drilling, taping, rasping, broaching and/or reaming is conducted on the bony surface of each facet joint 20 until subchondral bone of each spinal facet joint 20 is exposed.

It should be appreciated that preparation of the bony surface of each spinal facet joint 20 is deliberate so that the enlarged spinal facet joint space is specifically shaped to receive the specifically shaped implant 22. For example, if the implant is frusto-conical in shape, a similarly frusto-conical enlarged milled joint shape space will be produced. Measuring tools to measure the size of the spinal facet joint space 21 may be used such as a calliper and/or depth gauge to ensure the spinal facet joint space 21 is correctly sized for the associated implant. A trial implant may be located within the spinal facet joint space to determine if the spinal facet joint space 21 has been adequately prepared or alternately if a correctly sized implant has been chosen.

The ability to customize a spinal facet joint space 21 with preparation of the bony surfaces to receive an implant 22 remains essential to the appropriate selection of an interposition facet joint implant 22 that may be either the same or a different size at each spinal facet joint pair level, depending upon that patients individual anatomy, size and possible spinal deformity.

Once the enlarged spinal facet joint space 21 has been produced and measured, the implant 22 is located onto the head 52 of implant tool 50 discussed previously. When this is completed, the flats sections 56 located on the outer ring 54 engage with the flat sections 34 located on the top of the body 30. Also, the boss 53 locates within the hollow cavity 31 of the body 30 which causes the depressible ball 55 to be located within one of the scraping holes located at the top of the body 30 to hold the implant 22 to the implant tool 50.

The implant 22 is then placed at the top of the surgically prepared spinal facet joints. The implant tool 50 is then used to drive the implant 22 into the surgically prepared spinal facet joints. This can be achieved by either using hand force or using a mallet to hit the handle of the implant tool 52. As there is a series of circumferential barbs 40 that extend around the body 40, a stepped feeling is fed back through the tool as each barb enters the surgically prepared spinal facet joint.

Once the implant 22 is located within the surgically prepared spinal facet joints as shown in FIG. 4C, the implant is rotated through between 45 to 90 degrees until the large fenestrations 33 are located on opposites sides of the joint. That is, the large fenestrations 33 are located adjacent the subchondral bone of the joint as shown in FIG. 4D. The large fenestrations 33 provide the growth of new bone through the device, between each bony surface of the facet joint. That is, the large fenestrations 33 assists in fusion of the spinal facet joint.

The rotation of the implant also causes the scraping faces 41 of the barbs 40 to scrape bone material from the spinal facet joint that passes through the scraping holes 42 into the hollow cavity 31. The additional bone material through this auto-grafting technique also assists in fusion of the spinal facet joint. Further, rotation of the implant assists in preventing removal of the implant 22 from the spinal facet joints.

Additional oesteoconductive agent such as autograft, allograft, bone mineral substitute is impacted within the hollow cavity 31. The end wall 32 on the implant 22 prevents the oesteoconductive agent from falling through the central hollow cavity of the implant 22 onto the underlying nerve root. The large fenestrations 33 located within the sides of the implant 22 allow direct contact of the oesteoconductive agent with the subchondral bone surfaces of the facet joint. Because bone growth is promoted when under compressive loads, the hollow cavity 31 can be packed with bone graft material to ensure that the bone graft material is compressed against the subchondral bone to ensure the best possible conditions for fusion to occur.

The frusto conical shape of the body 30 assists in maintaining contact between the two adjacent facet joints which is necessary to achieve good fusion. The barbs 40 assist in preventing unwanted removal and movement of the implant 22 which again essential for good fusion. The fenestrations 33 located within the implant allow bone growth through the body 30 yet again in order to achieve good fusion. The implant 22 is also structural in nature. That is, it cannot be substantially crushed and provides support to the spinal facet joint. Further, the implant provides distraction of the spinal facet joint.

Typically additional fixing devices such as the use of anterior interbody graft/cage/ramp fixation and/or posterior dynamic stabilization devices (pedicle screw based, or interspinous process based, or similar) are also utilised to at least temporarily or permanently stabilise the spinal facet joint to assist in fusion.

Further, any osteoinductive material and/or solution and associated carrier vehicles to augment the chances of a successful biological fusion is typically located adjacent the spinal facet joint. Such osteoinductive materials include BMP, OP1, bone marrow aspirate, and other autologous growth factors, including collagen sponges or similar delivery vehicles.

The procedure can combine the placement of posterolateral on-lay graft material between the transverse processes at the same spinal level to enhance fusion.

The procedure can combine the placement of interbody grafts or cages at the vertebral level being fused.

The spinal facet joints in the lumbar, thoracic, and cervical spine are relatively large surface areas of bone that normally load under compression in vivo, which is ideal for achieving bony fusion, with the use of implant once the cartilage and subchondral bone has been exposed. Removal of the cartilage surfaces and the subchondral bone leaves an enlarged spinal facet joint space that lends to an implant being inserted to share load in compression which is a normal biomechanical feature in standing, walking and even lying down.

The above spinal fusion surgery can be performed via minimally invasive surgery techniques that can reduce morbidity, save on patient hospital stays, and reduce associated complications.

The facet joint in the lumbar spine is on average 16 mm long and 14 mm wide and has an average surface area of 160 mm², assuming an ovoid shape. Retention of the bony co-planar spinal facet joint surfaces, or a specifically reciprocally milled shape, adds to biomechanical stability of the triple joint and load sharing between any additional implants. Further, bleeding bone surfaces under compression, with a suitable implant with large fenestrations is likely to have a high fusion rate.

A distractive force may be applied to the facet articular processes either by patient positioning in a forward flexed posture, distraction through the pedicle screw and rod construct, or via a distractive force between the spinous processes at the level(s) being fused. Such a spinal facet joint interposition implant technique can exist without additional distraction of the spinal facet joint.

Each patient has slightly different anatomical features with regards their spinal facet joints with regards size and shape, and there may even be variation between two facet joints at the one spinal level. Surgical customization of the prepared bone surfaces between two facet joint articular processes can enable the appropriate selection of an interposition facet joint implant.

The solid nature of the interposition facet implant adds to the load sharing between it and any pedicle screw construct posteriorly, or cage/graft anteriorly between the two vertebral bodies being fused.

Pre-operative planning of the facet joint is easily obtained with routine radiological investigations (CT, MRI) and hence allows an indication of the size of the graft/implant/device needed.

The spinal facet joint can be easily assessed for degrees of biological fusion after insertion of an interposition facet implant using radiology techniques such as CT, MRI, and X-ray.

By having a known size of interposition facet implant, the surgeon will now have the ability to compare surgical techniques between patients and therefore permit more generalizable techniques that can be more easily scientifically compared.

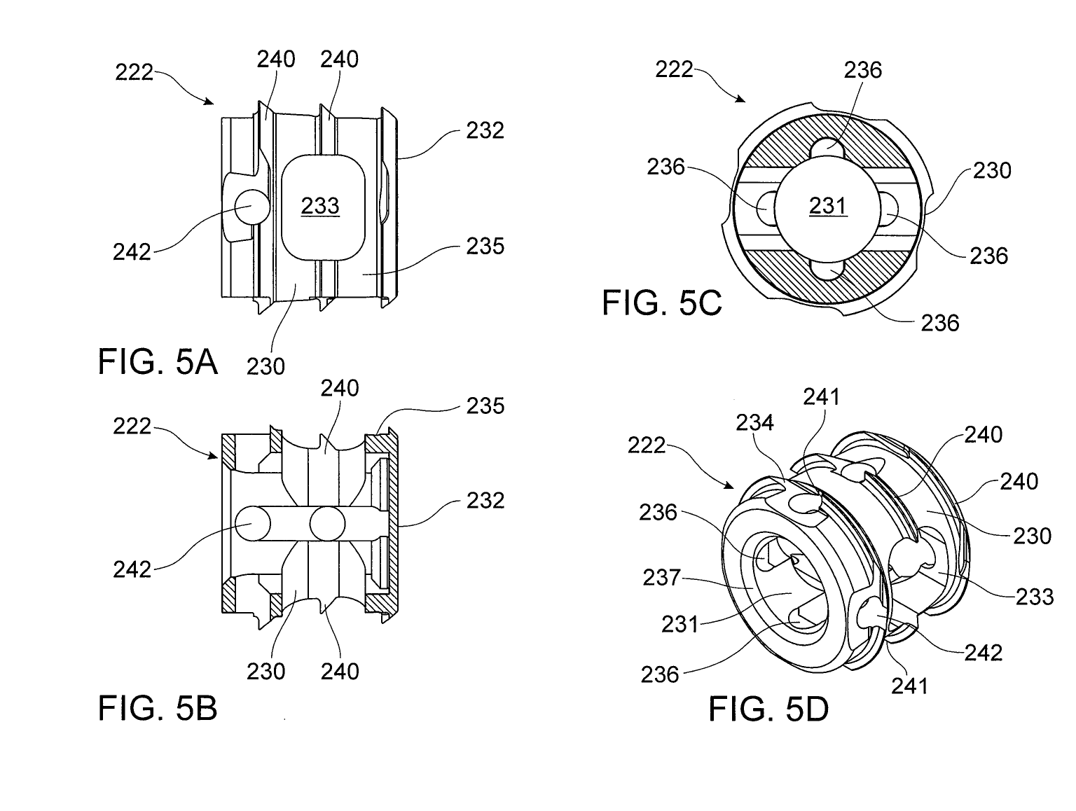

FIG. 5A to 5D show a second embodiment of a spinal facet joint 220. The spinal facet joint implant 222 is similar to the spinal facet joint implant 22. The spinal facet joint implant 222 is implanted in the same manner and using the same implant tool 50 as described above.

The implant 222 includes body 230 which is frusto conical in shape. A hollow central cavity 231 extends through a centre of the body 230. An end wall 232 is located adjacent the end of the body 230. A skirt 235 extends outwardly from the end wall 232 and extends around the circumference of the end wall 232 to form a well 238. Large fenestrations 233 extend through the body 230. The large fenestrations 233 have an external surface area ratio of approximately 45% of the total external surface area of implant 222. Four channels 236 extend along the length of an internal wall of the spinal facet joint implant 230. The four channels all extend into the well 238 located adjacent the end of the implant 222.

A series of circumferential, spaced-apart, barbs 240 are located along the length of the body 30. Each barb 240 has a scraping face 241 which is able to engage with the spinal facet joint. A series of scraping holes 242 are located adjacent some of the scraping faces 241 of some of the barbs 240. The barbs 240 are shaped so that rotation of the barb 240 will cause the scraping face 241 to engage or scrape the spinal facet joints upon rotation of the body 230.

A number of flat sections 234 are located adjacent the top of the body 230. The flats sections 234 are used to rotate the body 230.

A ledge 237 extends around the top of the spinal facet joint implant 222.

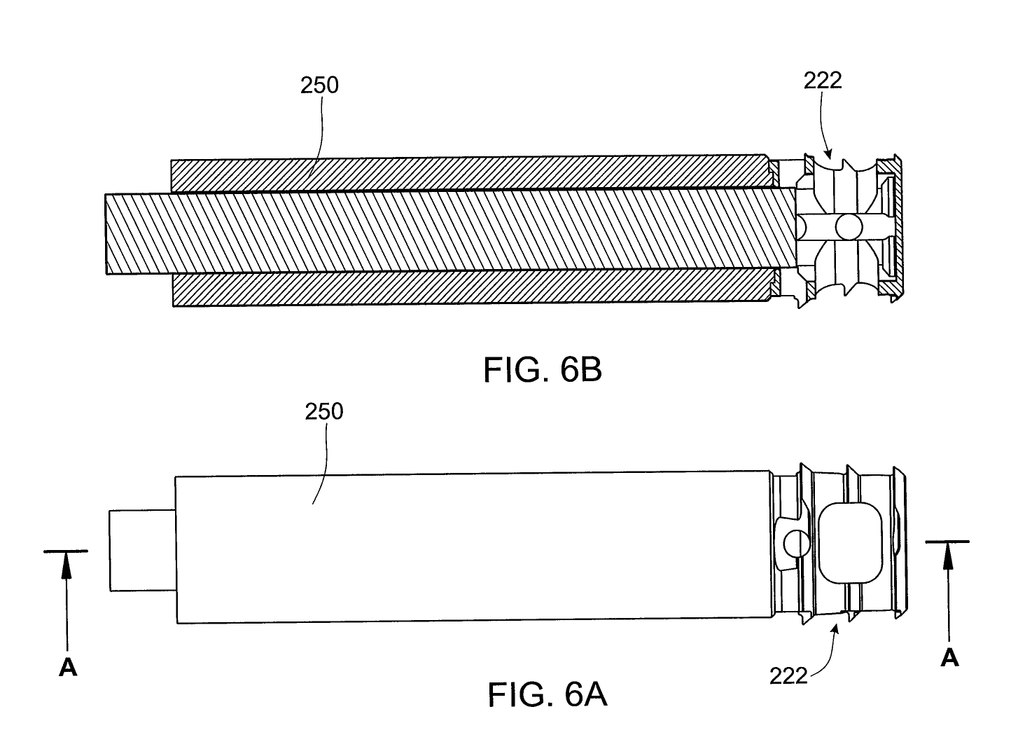

In use, a portion of oesteoconductive agent such as autograft, allograft, bone mineral substitute is located within the hollow cavity 232 of the body 230. The spinal facet joint implant 222 is then implanted using the implant tool 50 as described above. Additional oesteoconductive agent is then located within the hollow cavity 232. As is shown in FIGS. 6A and 6B, an impacting tool 250 is then placed within the ledge 237 of the spinal facet joint implant 222. This impacting tool 250 is used to impact and compress the oesteoconductive agent within the hollow cavity 231. This causes the oesteoconductive agent to pass through the large fenestrations 233 and contact the subchondral bone surfaces. This procedure creates compression which promotes bone growth.

The well 238, located adjacent the end of the body 230, traps oesteoconductive and osteoinductive agent and assists in preventing oesteoconductive and osteoinductive agent from falling onto the underlying never root. The four channels 236, which are in communication with the well 238, permit passage under suspected capillary action of liquid osteoinductive agent from the well 238 through the large fenestrations 233 and/or the scraping holes 242 onto the subchondral bone surface. This again will promote bone additional growth.

The spinal facet joint implant 222 provides a number of advantages. The spinal facet joint implant 222 can be inserted into a milled bone hole in the spinal facet joint. The milled bone hole may take a highly variable range of trajectories relative to the plane of the articular surfaces of the spinal facet joint. The trajectories that range from parallel to the articular surface of the joint through to perpendicular to the articular surface of the joint. This permits a forgiving and “safe” milling trajectory for the surgeon based upon the patient's anatomy, the approach being used, and ensures the benefit of removal of cartilage and bone for grating purposes, and affords the biomechanical effect of the spinal facet joint implant 222 as like a traditional trans-facet screw.

The body 230 of the spinal facet joint implant 222 has two large fenestrations to permit the ease of passage of both osteoconductive and osteoinductive graft materials to be in contact with the subcondral bone of the milled bone hole.

The spinal facet joint implant 222 has an associated impacting tool 250 that mates with the ledge to allow for osteoconductive and osteoinductive graft material impaction. This permits ease of insertion of the impacting tool 250 (especially for MIS usage) for in-situ grafting, after implantation of the spinal facet joint implant 222 into the bone hole.

The spinal facet joint implant 222 has a series of complete circumferential reversed angle barbs 240 on the external wall of the device. These prevent backing out of the device after implantation.

The spinal facet joint implant 222 has a series of incomplete reserved angle barbs 240 on the external surface of the device. These prevent backing out of the device after implantation

The spinal facet joint implant 222 has series of obliquely angled surfaces on the complete circumferential reversed angle barbs 240. These oblique angled surfaces act as scraping surfaces against the bony side walls of the milled facet joint hole when the implant is rotated after implantation. In such a way, the implant acts to “auto-harvest” bone graft from the side walls of the milled facet joint hole.

The spinal facet joint implant 222 has a series of oblique anti-rotation faces on the barbs 242 that are intentionally designed to resist rotation of the spinal facet joint implant 222 once it is inserted into the bony facet hole. This feature accounts for the variable torque moments that the spinal facet joint implant 222 is susceptible to from the circumferential side bony side walls that surround the spinal facet joint implant 222. This feature aims to minimize micro-motion of the spinal facet joint implant 222 and hence increase fusion rates of the facet joint bone side wall to the graft contents of the cage and to the aluminium oxide blasted walls of the spinal facet joint implant 222 which induce bone on-growth.

The spinal facet joint implant 222 has a series of scraping holes 242 located immediately adjacent to the obliquely angled surfaces on the incomplete circumferential reversed angle barbs 240. These holes act to receive bone that is auto-harvested from the bony side walls of the milled facet hole. This feature aims to enhance fusion rates by the improved delivery of fresh autograft to the combined contents of the spinal facet joint implant 222

The spinal facet joint implant 222 has a solid end wall 232 and a skirt 235. That is, the external walls of the spinal facet joint implant 222 rise from the end wall 232 to form a well 238 that contains both solid osteoconductive graft material and especially fluid osteoinductive substances (bone marrow aspirate, bone morphogenic protein, or similar).

The spinal facet joint implant 222 has four channel 236 on the internal side wall of the spinal facet joint implant 222 that permits passage/capillary action of fluid from the well 238 of the spinal facet joint implant 222 upwards to the scraping holes 242 in the body of the spinal facet joint implant 222 that are exposed for auto-grafting and bone through-growth. The fluid may also pass from the well like 238 along these side wall channels by direct pressure after osteoconductive graft material is plunged into the spinal facet joint implant 222 and the liquid component (bone marrow, bone morphogenic protein) is driven upwards from the well 238 of the spinal facet joint implant 222 along these channels 238 to the scraping holes 242.

It should be appreciated that various other changes and/or modifications may be made to the embodiments described without departing from the spirit or scope of the invention.

1. A method of performing surgery to enable joint fusion the steps including:
preparing bony surfaces of a joint to create an enlarged space between sides of the joint in which subchondral bone of the joint is exposed;
inserting a hollow structural implant, having at least two large fenestrations which are located on substantially opposite sides of the implant, into the enlarged space so that the implant contacts the subchondral bone; and
orientating the implant so that the large fenestrations are located adjacent the subchondral bone on respective sides of the joint.

2. The method of claim 1 including the step of filling the implant with an oesteoconductive agent so that the oesteoconductive agent contacts the subchondral bone surfaces through the large fenestrations.

3. The method of claim 1 including the step of placing a sponge filled with osteoinductive agent with the implant.

4. The method of claim 3 including the step of compressing the sponge within the implant.

5. The method of claim 1 including the step of manually and/or mechanically preparing the bony surfaces of the spinal facet joint by burring, drilling, taping, rasping, broaching and/or reaming the spinal facet joint.

6. The method of claim 1 including the step of milling bony surfaces of a joint to create an enlarged space between sides of the joint in which subchondral bone of the joint is exposed.

7. The method of claim 1 including the step of moving the patient to a surgical position to distract the joint.

8. The method of claim 1 including the step of inserting the implant with a driving force.

9. The method of claim 1 including the step of inserting the implant with a rotational force.

10. An implant able to be inserted into a surgically prepared joint space, the implant including:
a body having at least one large fenestrations extending through the body; and
at least one barb extending outwardly from a periphery of the body.

11. The implant of claim 10 wherein the implant the body of the implant is frusto conical in shape.

12. The implant of claim 10 wherein the body has a hollow central cavity.

13. The implant of claim 10 wherein the body has an end wall.

14. The implant of claim 10 wherein the body includes a skirt that extends around the body adjacent the distal end of the body.

15. The implant of claim 10 wherein the body has at least two large fenestrations which are located on substantially opposite sides of the body.

16. The implant of claim 10 wherein the at least one fenestrations is sized to have an external surface area of at least 35% of the total external surface area of the implant.

17. The implant of claim 10 including scraping holes are located through the body adjacent the barb.

18. The implant of claim 10 wherein the barb is shaped to scrape bone material a hollow central cavity of implant.

19. The implant of claim 10 wherein one or more channels extend along an internal wall of the body.
